(19) United States
(12) Patent Application Publication
(10) Pub. No.: US 2023/0106900 A1
(43) Pub. Date: Apr. 6, 2023

(54) Protection From Ionizing Radiation

(71) Applicant: Avisa Myko, Inc., San Francisco, CA (US)

(72) Inventors: Seshagiri RAGHUKUMAR, Dona Paula (IN); Ravindra ADGULWAR, Thane (IN); Swaraj Priyaranjan KUNAL, Thane (IN); Pratik Vinayak GHOLKAR, Kolhapur (IN); Kyatanahalli Srinivasa NAGABHUSHANA, Bengaluru (IN); Murali SASTRY, Pune (IN)

(21) Appl. No.: 17/760,000

(22) PCT Filed: Feb. 5, 2021
(86) PCT No.: PCT/US21/16966

§ 371 Date: Aug. 2, 2022

Related U.S. Application Data
(60) Provisional application No. 63/000,273, filed on Mar. 26, 2020.

(30) Foreign Application Priority Data
Feb. 6, 2020 (IN) 202011005142

(51) Int. Cl. A61Q 17/04; A61K 8/72; A61K 9/14; A61K 36/06; A61K 31/775; A61P 39/06

(52) U.S. Cl. CPC A61Q 17/04; A61K 8/72; A61K 9/14; A61K 36/06; A61K 31/775; A61P 39/06

(57) ABSTRACT

Compositions and methods for making and using a water soluble melanin, eumelanin, pheomelanin, allomelanin and DHN-melanin pigment produced from a fungus. The water soluble melanin can be used in a radioprotective composition having at least 0.1 to at least 8.0% (wt./vol.) melanin(s), a solvent and optionally a binder(s) and/or an additive(s). The aqueous soluble melanin has uses in medical, aeronautical, industrial, cosmetic and other applications that are disclosed.

### FIELD

The present teachings relate to compositions, methods of producing and using water soluble melanin for shielding and protection against UV-A, UV-B, X-rays and gamma radiation. The melanin produced can be in the form of a powder, a nanoparticle, a particle, and in slurries and solubilized in solutions thereof, The water soluble melanin can be used as protective: coatings, layers, within films and sprayed powders in various applications.

### BACKGROUND

Melanin is a pigment found in animals, plants, fungi and bacteria. It can also be synthesized from melanin precursors, for example, by oxidation of tyrosine, catalyzed by tyrosinase and followed by polymerization. Melanin contributes to the tolerance of black microorganisms to high levels of ionizing radiation including ultraviolet, X-ray and gamma radiations by absorbance of electromagnetic radiation (EMR) and dissipating the energy as free electrons that can be sequestered within the pigment and/or releasing the energy in the form of heat and/or conducted (H. Z. Hill, Bioessays (1992) 14:49-56).

Although melanin is readily found in various organisms including bacteria and fungi, the production and utilization of melanin for medical, aeronautical, industrial, cosmetic and other applications remains a challenging and poor economic proposition.

Therefore, there remains a need for creating and producing melanin compositions for use in radioprotective solutions against ionizing radiation that are convenient, economical to implement and amenable to bulk manufacture.

### INCORPORATION BY REFERENCE

All publications, references, patents, and patent applications mentioned in this document are herein incorporated by reference to the same extent as if each individual publication, reference, patent, or patent application was specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Reference will now be made in detail to certain claims of the invention, examples of which are illustrated in the accompanying drawings. While the invention will be described in conjunction with the enumerated claims, it will be understood that they are not intended to limit those claims. On the contrary, the invention is intended to cover all alternatives, modifications, and equivalents, which can be included within the scope of the invention as defined by the claims.

### BRIEF DESCRIPTION OF THE DRAWINGS

FIG. 1 illustrates a digital radiograph of Petri dishes, in triplicate, filled with dried acrylic paint, containing 0, 0.5, 1.0 and 1.5 mg melanin/cm² and exposed to X-ray of 30 keV.

FIGS. 2A-2D illustrate the shielding of paint containing 364 mg/cm² melanin when exposed to X-ray (40 keV).

FIG. 2A-2B, Control viewed with incandescent light and a digital image after 40 keV X-ray exposure, respectively.

FIG. 2C-2D, paint containing melanin viewed with incandescent light and a digital image after 40 keV X-ray exposure, respectively.

FIG. 3A-3B shows evaluation of acrylic paint and water with and without water-soluble melanin for shielding and transmission of 80 keV X-rays.

FIG. 3A depicts bar graphs of percent relative transmission of 80 keV X-rays through acrylic paint without and with 364 mg melanin/cm².

FIG. 3B depicts bar graphs of percent relative transmission of 80 keV X-rays through water without melanin and in a 4% melanin solution.

FIGS. 4A-4D illustrate the protective, shielding effect of dried melanin on seeds as determined by generation of a germination index.

FIG. 4A illustrates seeds shielded with a melanin paint at concentrations of 1.0, 2.0 and 3.0 mg/cm² melanin or unshielded (control) prior to X-ray or gamma-ray exposure. The top and bottom left dishes depict the top and bottom of a Petri dish coated with melanin, bottom dish contained seeds, while the top and bottom right Petri dish halves were without a melanin paint coating and contained seeds.

FIG. 4B is a bar graph of Paddy seed germination index as a function of n mg/cm² melanin for five increasing levels of radiation exposure.

FIG. 4C is a bar graph of Mustard seed germination index as a function of n mg/cm² melanin for five increasing levels of radiation exposure.

FIG. 4D is a bar graph of Moong seed germination index as a function of n mg/cm² melanin for five increasing levels of radiation exposure.

FIG. 5A-5B are digital images of Petri dishes exposed to 40 keV.

FIG. 5A illustrates a Petri dish containing 3 mm thick dried water-based glass paint not containing melanin. The darker color compared to 5B illustrates transmission of X-ray through the dish.

FIG. 5B illustrates a Petri dish containing 3 mm thick dried water-based glass paint containing 270 mg/cm². The lighter color compared to 5A illustrates less transmission and blocking of X-ray through the dish.

FIG. 6A-6B are digital images of a gel made with plain water (FIG. 6A) or with water containing 0.66% melanin at 3.0 mg/cm² (FIG. 6B) and exposed to 80 keV X-ray. The lighter color of FIG. 6B compared to FIG. 6A illustrates less transmission of X-ray and more blocking of X-ray through the gel.

FIG. 7 depicts the solubility of melanin in water at pH 4.0 to 14.0 and showing that the solubility increases as pH increases.

FIG. 8 illustrates DLS analysis of the size range of nanoparticles dispersed in water that were generated from dissolved melanin.

### DETAILED DESCRIPTION OF VARIOUS EMBODIMENTS

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting.

Disclosed herein are embodiments for producing water soluble melanin, compositions comprising water soluble melanin and methods for using water soluble melanin to protect against UV-radiation, X-rays and gamma radiation.

### Definitions

Unless defined otherwise, all technical and scientific terms used herein have the same meaning as commonly understood by one of ordinary skill in the art to which this invention belongs. As used herein, the recited terms have the following meanings. The following definitions are included to provide a clear and consistent understanding of the specification and claims. Any methods and materials similar or equivalent to those described herein can be used in the practice or testing of the disclosed invention, as it will be understood that modifications and variations are encompassed within the spirit and scope of the instant disclosure.

References in the specification to “one embodiment,” “an embodiment,” “another embodiment,” and the like, indicate that the described embodiment can include a particular aspect, feature, structure, moiety, or characteristic, but every embodiment may not necessarily include the particular aspect, feature, structure, moiety, or characteristic. Moreover, such phrases are not necessarily referring to the same embodiment. Further, when a particular aspect, feature, structure, moiety, or characteristic is described in connection with an embodiment, it is submitted that it is within the knowledge of one of ordinary skill in the art to effect or connect such aspect, feature, structure, moiety, or characteristic in connection with other embodiments whether or not explicitly described.

It is further noted that the claims may be drafted to exclude an optional element. As such, this statement is intended to serve as antecedent basis for the use of exclusive terminology, such as “solely,” “only,” “other than”, and the like, in connection with any element described herein, and/or the recitation of claim elements or use of “negative” limitations.

The term “and/or” means any one of the items, any combination of the items, or all of the items with which this term is associated. The phrases “one or more” and “at least one” when read in context of its usage are readily understood by one of skill in the art, particularly. For example, the phrase can mean one, two, three, four, five, six, ten, 100, or any upper limit approximately 10, 100, or 1000 times higher than a recited lower limit.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range as if each numerical value and sub-range is explicitly recited. As a non-limiting example, each range discussed herein can be readily broken down into a lower third, middle third and upper third, as well as nested ranges within a larger range, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges.

For example, a range of “about 1% to about 5%” or “about 0.1% to 5%” should be interpreted to include not just about 1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The term “about” as used herein can allow for a degree of variability in a value or range, for example, within 10%, within 5%, or within 1% of a stated value or of a stated limit of a range. For example, “about 50” percent can in some embodiments carry a variation from 45 to 55 percent. In yet another example, “about 10.0 wt. %” can be between 9.5 wt. % and 10.5 wt. %.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, element, the composition, or the embodiment. The term about can also modify the end-points of a recited range as discuss above in this paragraph.

In this document, the terms “a,” “an,” or “the” are used to include one or more than one unless the context clearly dictates otherwise. Thus, for example, a reference to “a component” includes a plurality of such components, so that a component Z includes a plurality of components Z. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; Information that is relevant to a section heading may occur within or outside of that particular section.

In the methods or processes described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing A and a claimed step of doing B can be conducted simultaneously within a single operation, and the resulting process will fall within the literal scope of the claimed process.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “attach” as used herein can refer to the claimed radioprotective composition, containing melanin, that can be applied, adhere, absorb, penetrate and/or coat intra- and/or inter-woven into a material, including but not limited to, a metal, a naturally sourced or synthetic textile, fabric, rubber, latex or edible produce.

The term “coating” as used herein can refer to the claimed radioprotective composition, containing melanin, that can be applied to a surface, mixed within or into a material, intra- and/or inter-woven into a material, including but not limited to a metal, a naturally sourced or synthetic textile, fabric, rubber, latex or edible produce. The coating can be in in the form of a paint, dye, gel, spray, paste, foam, liquid, cream, lotion, sizing agent, water-proofing agent. Moreover, the melanin, can also be added to, incorporated into or formulated into the material.

The final concentration of melanin can be in a range from about 1.0 mg/cm² to about 390 mg/cm². The wet or dried melanin concentration in the coating when either applied to a surface, mixed within or into a material, intra- and/or inter-woven into a material can be about 1.0 mg/cm², about 3.0 mg/cm², about 5.0 mg/cm², about 10 mg/cm², about 15 mg/cm², about 20 mg/cm², about 30 mg/cm², about 40 mg/cm², about 50 mg/cm², about 60 mg/cm², about 70 mg/cm², about 80 mg/cm², about 90 mg/cm², about 100 mg/cm², about 125 mg/cm², about 145 mg/cm², about 175 mg/cm², about 200 mg/cm², about 233 mg/cm², about 266 mg/cm², about 270 mg/cm², about 275 mg/cm², about 300 mg/cm², about 325 mg/cm², about 364 mg/cm², about 390 mg/cm², and about 400 mg/cm².

The term “ionizing radiation” as used herein can refer to electromagnetic wavelengths from 2×10⁻⁷ to 10⁻⁸ metres (Ultraviolet), 10⁻⁸ to 10⁻¹² metres (X-ray) and 10⁻¹² metres and up (Gamma ray) corresponding to energy levels of approximately 5 electron volts to several kiloelectron (kEv) and megaelectron volts (mEv).

The term “material” as used herein can refer to both raw materials and finished materials and the use of the finished materials in various domestic, commercial, medical, industrial, military and intra-galactic applications. Materials can refer to metals, a naturally sourced textile, including but not limited to a silk, wool, flax, cotton, hemp, linen, jute, papyrus, paper, bamboo, leather, latex, rubber, glass, asbestos, a synthetic textile including but not limited to a polyester, nylon, acrylic, rayon, neoprene, polypropylene, plastic, nitrile rubber, latex; edibles including but not limited to rubber, gum, fruit, vegetable, edible produce as well as a window treatment, a floor covering, a furniture component, clothing, gear, rigging, fabric-including but not limited to drapery, upholstery, decorative, bedding; exterior paint, dye, interior paint, radiation shielding paint; boat and vehicle exterior surfaces, including but not limited to, flotation devices, bumpers, convertible tops, canopies, door-edge guards, tires, and interior surfaces, including but not limited to, dashboard, rear window shelf and seats and seat coverings and padding, door panels, ceiling coverings, flooring, and the like.

The term “edible produce” as used herein can refer to fruits, vegetables, sprouts, grasses, seeds, nuts, oats, and grains.

The disclosed water soluble melanin is produced by a filamentous fungus identified as a species of the genus Gliocephalotrichum. A culture of the fungus has been deposited under the Budapest Treaty at the International Depository Authority of the Microbial Type Culture Collection, CSIR-Institute of Microbial Technology, Chandigarh, India. The culture has been assigned the number MTCC 5489.

The terms “radioprotective” and “radioprotection”, can be used interchangeably and as used herein refer to a material, clothing, barrier or coating containing melanin that absorbs, protects and/or precludes penetration of ionizing radiation into an animal's body or appendage(s), or permits dissemination and/or escape of radiation from an enclosure, chamber, instrument, room and the like and into the exterior spaces and/or environment.

The present teachings can be implemented in the creation of a radioprotective melanin composition. The composition can have about 0.1 to about 3.0% to about 99.0% melanin and values within these amounts. The melanin can be eumelanin, pheomelanin, allomelanin, DHN-melanin and combinations thereof. Eumelanin can be derived from tyrosine as a precursor. Precursors of tyrosine and cysteine can produce Pheomelanin while Allomelanins are formed from nitrogen-free precursors such as catechol, 1,8-dihydroxynaphthalenes (DHN). DHN-melanin is produced by bacteria and fungi, including pathogenic Aspergillus sp. Table 1 compares the various sources of melanin pigments. Further details on the origins and types of melanin can be found in M. d'Ischia et al. Pigment Cell Melanoma Res. (2013) 5:616-633 and M. d'Ischia et al. Pigment Cell Melanoma Res. (2015) 5:520-544.

TABLE 1

| | | | | DHN-melanin |
|---|---|---|---|---|
| Source | Eumelanin | Pheomelanin | Allomelanin | Pyomelanin |
| Animal | √ | √ | | |
| Fungal | √ | √ | | √ |
| Bacterial | √ | | | √ |
| Plant | | | √ | |
| Synthetic | √ | √ | √ | |

Melanin can be produced in a laboratory or at an industrial scale in a fermentation tank/bioreactor. The microorganism can secret melanin into the liquid culture medium/broth or directly synthesized by the fungus extracellularly in the culture broth. Melanin can be directly used from the liquid culture medium solution as the source of dissolved melanin and used for purposes described herein, as well as formulated directly into nanoparticles of melanin for uses described herein. The yield of melanin produced at either laboratory or industrial scale can be about 4.0 gm/L, 5.0 gm/L, 6.0 gm/L, 7.0 gm/L, 8.0 gm/L, 9.0 gm/L, and up to 10.0 gm/L.

Alternatively, from the broth solution, melanin can be precipitated as a fine powder, particles or formulated directly into nanoparticles of melanin. There are many forms melanin can take and methods for its production. Melanin can be derived; (1) Directly from the culture broth as a solution; (2) spray-dried as a fine powder (about 1 micron to about 100 microns) and resolubilized in water as required; (3) acid-precipitated as a fine powder and resolubilized in a base when required; and (4) Melanin solution from (1) as above or after conversion into nanoparticles (about 10 nm to about 300 nm). The form of melanin produced provides unlimited opportunities for incorporating melanin into ionizing radiation protective applications, materials, products and enclosures.

Applicants have developed efficient and economical processes to produce nanomelanin with nano-particle sizes ranging predominantly from about 80 nm to about 100 nanometres across a size range of about 10 nm to about 300 nm. The produced melanin nanoparticles can have a size from about 20 nm to about 200 nm, from about 20 nm to about 100 nm, from about 20 nm to about 150 nm, from about 20 nm to about 175 nm, from about 10 nm to about 400 nm and from about 10 nm to about 500 nm. The nanoparticles could be generated in an aqueous phase and dispersed in water, or generated in an organic solvent phase and dispersed in an organic solvent.

conversion into nanoparticles (about 10 nm to about 300 nm)

There are many different melanin precursors, including, but not limited to: One or more of tyrosine, L-dopa (3,4-dihydroxyphenylalanin), D-dopa, catechol, 4-aminocatechol 5-hydroxyindole, 5,6-dihydroxyindoline, 6-hydroxyindole, N-methyl-5,6-dihydroxyindole tyramine, dopamine, tyrosine, cysteine, m-aminophenol, o-aminophenol, p-aminophenol, 2-hydroxyl-1,4-naphthaquinone, 4-metholcatechol, 3,4-dihydroxynaphthalene, gallic acid, resorcinol, 2-chloroaniline, p-chloroanisole, 2-amino-p-cresol, 4,5-dihydroxynaphthalene, 1,8-dihydroxynaphthalene, 2,7-disulfonic acid, o-cresol, m-cresol, p-cresol, combinations thereof and the acid-addition salts thereof.

The radioprotective composition can also comprise 0.1 to 97% of a solvent including, but not limited to, water, dichloromethane (DCM), N-methyl pyrrolidone, tetrahydrofuran (THF), ethyl acetate (EtOAc), acetone, dimethylformamide (DMF), acetonitrile (MeCN), dimethyl sulfoxide (DMSO), nitromethane, propylene carbonate, formic acid, n-butanol, isopropanol (IPA), nitromethane, n-Propanol, ethanol, methanol, acetic acid, nitrocellulose lacquer, and combinations thereof. For reference, but not to be limiting, a highly polar solvent has a dielectric constant greater than 5.0 and can be either a polar aprotic or polar protic solvent.

Potential non-polar solvents can include, but are not limited to: hexane, benzene, toluene, 1,4-dioxane, chloroform, diethyl ether, mineral spirits (US term) aka white spirits (UK term), acetone, turpentine, naphtha, toluene, methyl ethyl ketone (MEK), dimethylformamide (DMF), 2-butoxyethanol, glycol ethers, glycerol, sugar alcohols, as known to one of skill in the art.

Additionally, the water soluble melanin radioprotective composition can optionally have 0.1 to 30% of one or more binders and 0.1 to 50% of an optional additive. Examples of envisioned binders include, but are not limited to an alkyl resin, an acrylic resin, a vinyl-acrylic resin, a vinyl acetate/ethylene (VAE) resin, a polyurethane, polyester, a melamine resin, epoxy, silanes, siloxane or oil and combinations thereof as known to one of skill in the art. Mordants are also included as binder and can include, but are not limited to sodium chloride, polyvalent metal ions of aluminum, copper, iron, iodine, potassium, sodium, as well as tannic acid, alum, and chrome alum and combinations thereof as known to one of skill in the art. Envisioned additives can include, but are not limited to additives that affect pigment stability, antifreeze properties, viscosity adjustors and/or controls foaming and combinations thereof.

The water solubility of the disclosed melanin composition can be readily tested by demonstration of the Tyndall effect. Simply described, the up to 1% solution of the disclosed melanin dissolved in water or another solution, when exposed to a light beam, the wavelengths of the light remains unreflected and did not scatter in a multitude of directions within the solution. This is because the melanin was dissolved in the water. If it were not dissolved in the water but rather, a dispersion of minute particulates, the various wavelengths of the light would be reflected off the particles and scattered within the solution. The solution might appear to be of a blue color, as the shorter blue wavelengths would be scattered by the non-soluble particles.

Moreover, the ability of tyrosinases to be upregulated during stress conditions and impart a survivability protective adaptation, including exposure to ionizing radiation, is seen in fungi able to produce melanin(s) (E. Selinheimo, et al. FEBS J. (2006) 273:4322-4335).

Viscosity Reducers

The viscosity of the disclosed radioprotective melanin composition can be a consideration when considering the method for applying the radioprotective composition to a required thickness and/or shape, speed of manufacturing and so on as would be known to the skilled artisan. An increase in speed of incorporating, wetting and/or layering/applying the radioprotective composition can be achieve by including a viscosity reducer in the radioprotective composition's formulation. Examples of materials that can have viscosity reduction effect depending on the melanin(s)' concentration and source(s) and selection of binder(s), used in the disclosed radioprotective composition(s) include, but are not limited to, sodium chloride, lithium chloride, potassium chloride, sodium sulfate, tetrasodium phosphate, Na₄P₂O7₄, and the decahydrate form, and sodium chloride with caustic soda. Viscosity reducers can be used alone and in combination from between 0-50 wt. % and ranges within this range in formulating the radioprotective composition depending upon the use and material's purpose and/or end use.

Anti-Foaming Agents

Agents that reduce or eliminate foaming (Defoamer agents) and preclude bubbles from forming in the disclosed radioprotective composition can help provide a coating of uniform appearance and eliminate potential weak points, pin holes or breakage prone areas in the finished material. Examples of anti-foaming agents are known to the skilled artisan and can include, but are not limited to oil-based anti-foam agents including, but not limited to oils insoluble in the foaming medium (except silicone oil), vegetable oil and mineral oil; powder-based anti-foam agents can be oil based defoamer surrounding/on a particulate such as silica and can be added to powdered compositions of detergent, plaster and cement; water-based anti-foam agents including, but not limited to sodium hydrogen carbonate, sodium carbonate, magnesium carbonate, potassium carbonate, and calcium carbonate and ammonium hydrogen carbonate, and ammonium carbonate; silicon-based anti-foaming agents such as polyethylene glycol and polypropylene glycol. In addition, auxiliary foaming agents can include calcium dihydrogen pyrophosphate, dipotassium hydrogen phosphate, disodium hydrogen phosphate L-ascorbic acid, L-aspartic acid, galacturonic acid, glucuronic acid, L-glutamic acid, monosodium fumarate, potassium L-bitartrate, potassium aluminum sulfate, potassium dihydrogen phosphate, sodium dihydrogen phosphate, and sodium DL-malate. The anti-foaming agent(s) can be used alone and in combination from between 0-5 wt. % and up to 10 wt. % and ranges within this range in formulating the radioprotective coating composition depending upon the use and product produced from the resulting coated material.

As indicated above, the melanin pigment can be isolated and/or derived from an animal, fungal, bacterial, plant or synthesized chemically.

Numerous animals and humans naturally have melanin in the cells of surface tissues including the skin, hair, animal hair and retina, Melanin is also found in feathers.

Fungal melanins are widely known and produce eumelanin, pheomelanin and DHN-melanin. Examples of melanin producing fungi include, but are not limited to, Gliocephalotrichum simplex, Gliocephalotrichum sp. MTCC 5489, Cladosporium spp., Curvularia spp., Chaetomium sp., Cryptococcus neoformans, Agaricus bisporus, and pathogenic fungi such as Aspergillus spp., Exophiala spp., Sporothrix schenckii, Alternaria alternata, Cladosporium carionii and Fonsecaea spp.

Many bacteria produce eumelanin. Examples of melanin producing bacteria include, but are not limited to, Pseudomonas sp., Brevundimonas sp., Vibrio cholerae, Streptomyces sp., recombinantly engineered Escherichia coli.

A further synthetic synthesis of melanin can be carried out using precursors of melanin supra. Eumelanins can be synthesized from tyrosine or dihydroxyphenylalanine as a precursor catalyzed by tyrosinase as well as derived from L-DOPA and are perceived as black or brown pigments. And synthetic replication of microbial melanin synthesis can be achieved via catalysis of phenoloxidases, such as tyrosinases, laccases or catacholases and also via the polyketide synthase pathway. The yellow or reddish melanins originating with homogentisic acid and catalyzed by tyrosinases are pyomelanins while DHN melanins, black or brown, are derived from acetate as a precursor in the polyketide synthase pathway (J. D. Nosanchuk, A. Casadevall, Cellular Microbiology, (2003) 5(4), 203-223).

Melanin is known for providing protection from ionizing radiation and attenuating ionizing radiation such as UV rays A, B and C, radioactive isotopes and environmental insults, e.g., temperature fluctuations, The disclosed radioprotective composition can be incorporated into the formulation of a material, including but not limited to, paint, dye, gel, spray, paste, foam, liquid, cream, lotion, dye, sizing agent, water-proofing agent, a synthetic textile including but not limited to a polyester, nylon, acrylic, rayon, neoprene, polypropylene, nitrile rubber. The radioprotective composition can be applied, impregnated, coated, covered, layered into or between, and interwoven into a naturally sourced textile, including but not limited to, a silk, wool, flax, cotton, hemp, linen, jute, bamboo, leather, latex, rubber, glass, and asbestos. These materials have a wide range of uses including, but not limited to, protection and/or attenuation of and from forms of ionizing radiations such as UV-A, UV-B, X-rays, gamma rays. Exposure is more acute to ionizing radiation in a high altitude environment, a high sunlight environment, a northern polar environment, a southern polar environment. Individuals aboard the ISS, astronauts, an airplane, airplane pilot, atop a mountain, uranium mines, radioactive isotope generation including, but not limited to, plutonium isotopes used in radioisotope thermoelectric generators, radioisotope heater units, as well as utilization in equatorial regions of Earth, and an ozone weakened and/or depleted atmosphere.

Melanin pigments can also provide protection against photo-bleaching, color fading, degradation of the colors in textiles and fabrics including but not limited to drapery, upholstery, decorative accessories, bedding, window treatment, draperies, carpet, rug, floor covering, a furniture component (seat, cushion, pillow, ottoman), clothing, apparel, gear, rigging, exterior paint, interior paint, dye, radiation shielding paint, boat and vehicle exterior and interior surfaces, including but not limited to, flotation devices, seat coverings and padding, door panels, seats, ceiling coverings, convertible tops, dashboard, rear window shelf and the like. Melanin pigments can also protect against oxidation and can provide a natural method for protecting produce against oxidation including, but not limited to, vegetables, nuts, fruits, grains, and legumes.

Melanin pigments can also be employed in cosmetics, sunscreen and therapeutics to preclude melanocyte formation within the skin, to preclude moles and as sunshield for preventing UV exposure to individuals afflicted with vitiligo, albinism and other depigmentation or hyperpigmentation conditions.

As indicated above, the melanin pigment can engulf, be mixed with metal powder and/or metal filings of a metal including, but not limited to, bismuth, lead, aluminum, copper, tungsten, iron and steel. Such a mixture can be sprayed, sprinkled on an adhesive applied to a protective garment, surface and the like and sealed in place to create a radioprotective garment, surface, enclosure or barrier.

The melanin layer deposited when applying, integrating or mixing the radioprotective composition into one or more materials or further components of a composition can impart about 1 mg/cm² of melanin, 3 mg/cm² of melanin, 5 mg/cm² of melanin, 10 mg/cm² of melanin or more, depending on the level of radioprotective attenuation desired. The energy level of the ionizing radiation will influence the amount of melanin pigment that can be layered, incorporated, applied to the material as well as the end use of the material.

Envisioned are melanin radioprotective compositions effective against ionizing radiation with energy levels ranging from 10 electron volts (eV) to 1×10⁶ mega electron volts (MeV). Such energy level can encompass UV rays, X-rays and gamma rays. The melanin radioprotective composition can achieve an effective attenuation coefficient ranging from about 10%, about 12%, about 14%, about 16%, about 18%, about 20%, about 25%, about 30%, about 35% about 40%, about 45%, about 50%, about 55%, about 60%, about 65%, about 70%, about 75%, about 80%, about 85%, about 90%, about 95%, 96%, 97%, 98%, 99% to about 100% of the ionizing radiation. This is further illustrated in Example 4 where seeds of various plants were placed in containers coated with a paint corresponding to 3 mg/cm² of melanin on the interior surface of the container. The container was used to protect against 1.33 MeV (1.33×10⁶ eV) and gave 100% protection to seeds as seen by their subsequent germination. The control seeds, conversely, were unable to subsequently germinate.

### Methods

Production of Melanin Pigment from Fungi

Melanin pigment was prepared in a bioreactor (up to at least 50 Litres) as is disclosed in Indian Patent No. 285818. Surprisingly, the inventors have further optimized melanin production levels making the generation of a water soluble, eumelanin pigment cost effective. They have achieved a melanin pigment produced at a cost below $0.20 USD per gram. Currently, commercial sources sell melanin pigment for over $400.00 USD per gram (derived from Sepia officinalis, Millipore Sigma, SKU M2649-1G) with synthetically produced melanin comparably priced as well.

Applicant has been able to substantially reduce costs by using inexpensive raw materials, purchasing raw materials in bulk quantities, using large bioreactor capacity for producing large amounts of fungal melanin from Gliocephalotrichum simplex, MTCC 5489. This fungus secretes up to at least 8 gm dry weight per litre of the water soluble melanin pigment into the bioreactor broth medium which exceeds standard melanin generation methods by at least 21%. The dissolved melanin in the liquid culture medium/broth can be spray-dried, freeze-dried, precipitated with acid into a powdered form, or converted into melanin nanoparticles or used as a melanin broth solution. Regardless of the final form of melanin it can be used in several differing aeronautical, shield, commercial, medical, therapeutic, textile, fabric, cosmetic and industrial applications as illustrated in the following Examples. Applicants have developed and implementation innovative and novel uses of melanin pigment to be both an attractive alternative to metal, lead and heavy shielding for protection from ionizing radiation and to also provide economical, efficient and commercial opportunities to use melanin to protect many lifeforms, materials and radiation barrier surfaces from ionizing radiation.

### EXAMPLES

### Example 1: Melanin Pigment in a Water Soluble Paint Against X-Rays

The disclosed radioprotective water soluble melanin pigment can provide attenuation of ionizing radiation following incorporation into a paint, dye, adhesive, gel, spray, paste, foam, liquid, cream, lotion, sizing agent, water-proofing agent and combinations thereof.

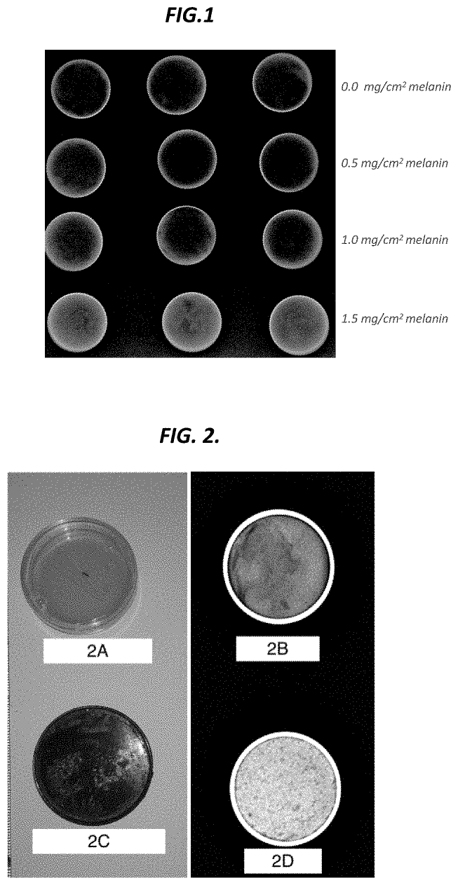

A 1% solution of soluble melanin (derived from MTCC 5489), corresponding to 1000 mg of melanin in 100 ml water was prepared. Volumes of 1.5 ml, 1.0 ml and 0.5 ml of this solution, containing a total of 15, 10 and 5 mg of melanin was mixed with 2.0 ml, 2.5 ml or 2.0 respectively, in triplicate, with a water soluble, commercially available, transparent fabric paint (Camel Brand, Kokuyo Camlin, Mumbai, India) and poured into the bottom of each 3.5 cm diameter Petri dish with an approximate surface area of 30 cm² and allowed to dry overnight at 60° C. This resulted in concentrations of 1.5 mg, 1.0 mg and 0.5 mg of melanin/cm² in each Petri dish. FIG. 1 illustrates the results. The control dishes had only paint and absent melanin and melanin concentrations were increased in each successive descending row. These coated Petri dishes were placed, in triplicate and exposed to X-ray levels commonly used for soft-tissue X-rays, approximately 40 keV. Digital images were obtained to see if the melanin-coated petri dishes provided any blockage of the X-rays. Evident in the control dishes were very dark black images indicating no blockage of X-ray radiation. The successive increasing melanin had a light white image on the X-ray film for 0.5 mg/cm² of melanin which increased in a whiter intensity with increasing levels, 1.0 and 1.5 mg/cm² melanin concentration.

These results indicate that with increasing levels of water soluble melanin pigment the melanin enhanced paint provided protection to the ionizing X-ray radiation. Such results can have a profound impact in areas were finger dexterity and freedom of movement is critical when handing isotopes. Such areas can include, but are not limited to, surgical procedures, nuclear power plants when moving, changing or replacing uranium rods and the like.

### Example 2: Qualitative Test to Determine Melanin Slurry Blocking of X-Rays

A qualitative test to determine blocking of X-rays was carried out as follows. Melanin, weighing 3.5 grams was mixed with 6 ml of an acrylic, water-based glass paint (Camel Brand, Kokuyo Camlin, Mumbai, India), to make a thick slurry. This was transferred to a of 3.5 centimeter diameter Petri dish by one cm depth to form a melanin-paint layer of 1.0 cm and a melanin concentration of 364 mg/cm². A control Petri dish contained glass paint without melanin at a dried thickness of 1.0 cm. The dishes were exposed to X-rays at an intensity as is used for soft tissue X-rays, about 40-44 keV and digital images were generated. A dark colour on the X-ray film represented X-ray transmission through the paint and film exposure, while a lighter white colour represented blocking of X-ray transmission when the paint contained melanin. As seen in FIG. 2B, the dish containing paint without melanin appeared dark in the X-ray image, while the dish with melanin paint, FIG. 2D, was almost white and so almost no film exposure to the X-rays, which is evidence of blocking of X-ray transmissions by paint containing melanin.

### Example 3: Quantitative Test to Determine Blocking of X-Rays by Melanin Containing Paint

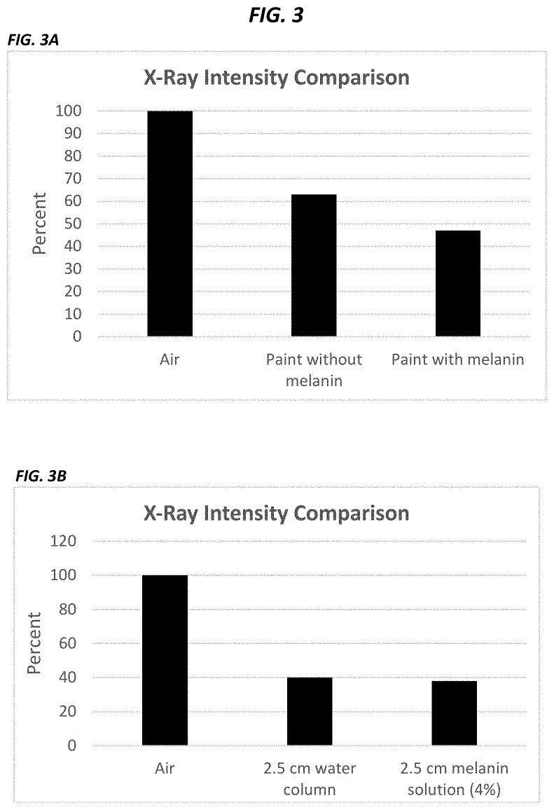

A quantitative estimation using 80 keV X-ray transmission was carried out to compare the relative transmission of X-rays through Air and Acrylic Paint with and without melanin and were prepared as was done for the qualitative test in Example 2. FIG. 3A depicts bar graphs of percent X-ray transmission through Air, 1 cm thick Acrylic Paint without melanin and 1 cm thick Acrylic Paint with 364 mg melanin/cm². The results demonstrate that Paint without melanin blocked 37% of X-rays (63% transmission) while paint with melanin blocked 53% of X-rays (47% transmission) for a 16% improvement in blocking X-ray penetration. FIG. 3B depicts bar graphs of percent X-ray transmission through Air, a 2.5 cm thick water column and a 2.5 cm thick water column containing a 4% melanin solution. The results for both FIGS. 3A and 3B show quantitatively that Paint with melanin transmitted the least level of X-rays and thus, paint containing melanin did effectively block X-ray transmission compared to either air or paint without melanin. Interestingly, the results also show that a 4% solution of Melanin had similar X-ray penetration and transmission as X-rays through plain water.

These experiments illustrate that a paint coating containing melanin was effective in blocking ionizing radiation. The melanin containing paint can be applied on the surface of a natural or synthetic fabric or textile, plastic, metal, or any suitable solid surface to enhance blocking of ionizing radiation.

An aqueous melanin solution was shown to poorly block X-rays. A 4% melanin solution at a depth of 4 cm in a container, with a melanin concentration of 125 mg/cm² blocked 62% of X-rays at 80 keV intensity. However, the same amount of plain water, without melanin, in a similar container also blocked X-rays to a similar extent and at the same intensity. Therefore, it can be inferred that melanin in the form of a solution was just as effective as water in blocking X-rays. While not wishing to be bound by theory, it appears that the paint, when containing melanin, either in dispersion (i.e. dispersed) or solubilized (i.e., in solution), might have acted as a melanin stabilizing agent thereby facilitating a potentially more efficient use of melanin to block penetration and transmission of X-rays into and through the melanin containing paint.

### Example 4: Melanin to Protect Life Forms

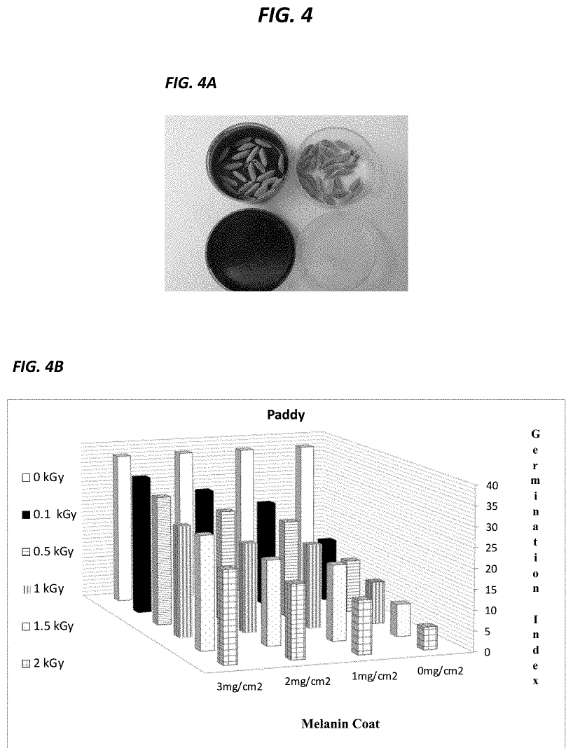

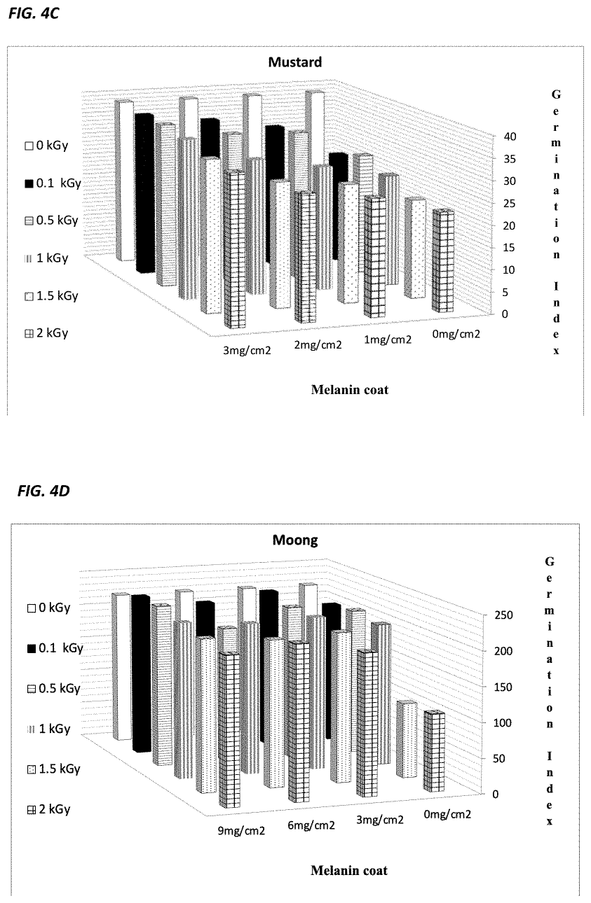

The bottom and the inside of the lid of 3.5 cm diameter Petri dishes were coated with a melanin paint in triplicate as described in Example 1. Thus, volumes of 3.0 ml, 2.0 ml or 1.0 ml of the solution containing a total of 30, 20 and 10 mg of melanin was mixed with 1.0 ml, 2.0 ml or 3.0 ml, respectively of a water soluble, commercially available, transparent glass paint and poured into the bottom of each Petri dish and allowed to dry overnight at 60° C. This resulted in concentrations of 3.1 mg, 2.1 mg and 1.0 mg of melanin/cm² in each Petri dish. Control dishes had only a coating of paint, without melanin, added to the bottom and the inside of the dishes. Seeds of paddy were placed inside the Petri dishes as shown in FIG. 4A, covered and exposed to different Gray levels (kGy) of Cesium 137 (Cs-137) gamma irradiation, corresponding to a radiation energy of 662 keV (FIG. 4B). Likewise, volumes of 3.0 ml, 2.0 ml or 1.0 ml of the solution containing a total of 10, 6.6 and 3.3 mg of melanin was mixed with 1.0 ml, 2.0 ml or 3.0 ml, respectively of a water soluble, commercially available, transparent glass paint and poured into the bottom of each Petri dish and allowed to dry overnight at 60° C. This resulted in concentrations of 3.0 mg, 2.0 mg and 1.0 mg of melanin/cm² in each Petri dish. Control dishes had only a coating of paint, without melanin, added to the bottom and the inside of the dishes. Seeds of mustard and green gram (moong) were placed inside the Petri dishes as shown in FIG. 4A, covered and exposed to different Gray levels of Cesium 137 (Cs-137) gamma irradiation, corresponding to a radiation energy of 662 keV. Results for Mustard seeds are shown in FIG. 4C and for Moong seeds in FIG. 4D.

The seeds were added to Petri dishes having: no paint shielding, only paint shielding or paint+melanin composition shielding, covered and were exposed to various levels of Gray (Gy) levels of gamma-rays. The germination index as a function of melanin/cm² for five increasing levels of gamma-rays are illustrated in FIGS. 4B-4D. Subsequent to exposure, the seeds were allowed to germinate on water-soaked blotting paper kept in a moist chamber. Based on the percentage germination, a germination index was prepared according to standard equations:

Germination index (GI)=(3*n1)+(2*n2)+(1*n1), where:

N1-n3 is Number of seeds germinated on Day 1, 2 and 3;

N=Total seeds germinated.

It was noticed that a high rate of seed mortality, seen as no seed germination, was observed in the Control Petri dishes which had an absence of melanin shielding. Alternatively, a high rate of survival, seen as percent germination under test conditions, resulted when the seeds were enclosed/shielded in paint+melanin covered plates following exposure to various levels of gamma-rays.

Preservation of crops and edible seeds against gamma radiation can be useful, for example, but not limited to, at least under conditions where gamma radiation would be expected to destroy seed germination potential, vitality and maturation. One practical application where preservation of seed vitality will be essential can be in space and intra- and inter-galactic travel. The seeds would be utilized for food production and germination, including, but not limited to, sprouting, sporeling, and pollen tube growth would be just one process in the production of nutritious foods as well as for growing them under artificial conditions (absence of gravity, natural sunlight and the like) either on the Moon, in an orbiting space station as well as within other extraterrestrial environments. It is noted that the term “seed” as used herein, can refer to seed plants, the spermatophytes, including the gymnosperm and angiosperm plants, as well as, for example, but not limited to, seed corn, sunflower seeds, seed potatoes as well as the egg, ovule, pollen, and sperm that, following fertilization, form a zygote which can be seen to develop through either germination or embryogenesis into a mature organism.

Significantly, this example demonstrates that life forms, in general, can be protected against the lethal effects of gamma radiation, remain viable, and can have the potential to be stored indefinitely for later use by shielding with melanin. The life forms can be in any one or more of a dormant, hibernating, induced coma and other states of inactivity and which are then able to return to viability when favorable conditions are present.

### Example 5: Powdered Melanin Thickness and the Percent of Radiation Blocked to Prevent Radiation Exposure

This Example illustrates the determination of the depth of melanin powder that can be used to achieve blocking a specific level of radiation.

In one embodiment, melanin can be dried as a coating on a solid surface or textile to provide shielding against ionizing radiation. Thus, a coating made exclusively of melanin powder applied to a depth of 1 cm and with melanin at a concentration of 271 mg/cm² also showed blocking of X-rays.

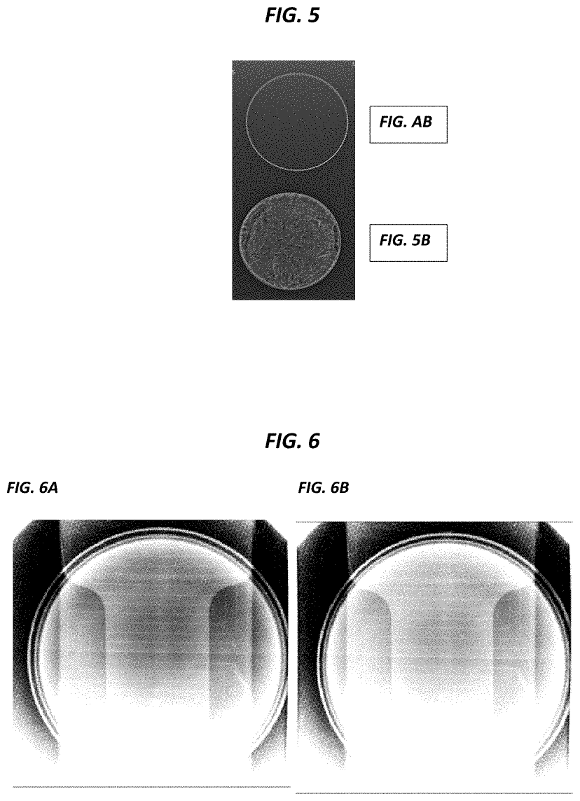

A total of 15.0 gm melanin was first dissolved in 1500 ml distilled water. The solution was concentrated by rotator evaporation to 50 ml. The solution was poured into a Petri dish of 8.4 cm diameter and air dried for 5 days. The melanin coating thickness was 3 mm. Based on the area of the Petri dish and the amount of melanin present, it was calculated that the Petri dish contained 271 mg/cm² of melanin. FIG. 5A depicts an empty Petri dish and FIG. 5B the Petri dish with the dried, 3 mm melanin coating. The empty Petri dish allowed transmission of X-rays (dark circle on the X-ray) while the Petri dish with 3 mm melanin blocked X-rays substantially (gray colored circle on the X-ray).

### Example 6: Layering Melanin “Sheets” by Adding Powdered Melanin to Increase the Percent of X-Ray Radiation Blocked Further Limits X-Ray Radiation Exposure

A 9 cm diameter Petri dish was filled with 40 ml of 0.66% melanin solution in a 1.0% gel, including but not limited to, a gel such as agar, carrageenan resulting in a melanin concentration of 3 mg/cm² at a gel thickness of 1 cm. The gel was allowed to form a semi-solid, the Petri dish was placed on an unexposed X-ray film and then exposed to 80 keV of X-rays. A 4% block of X-ray radiation was noticed in the presence of melanin only. As illustrated in FIG. 6A, no melanin was present in the gel and no X-ray blockage occurred (darker) while FIG. 6B shows the blockage of X-rays as seen by the heavy white density of the X-ray image after exposure.

This experiment illustrates the potential for X-ray shielding using a melanin containing gel that could be less bulky, of substantially less weight than lead shielding and would achieve comparable percent blocking of X-ray radiation. Too, the gel+melanin can be biodegradable and incorporated in small quantities to maintain the melanin concentration and provide radiation shielding for persons undergoing X-ray examination, radiation therapy, radon exposure in uranium and other radioactive mining settings, astronauts. The ionizing radiation exposure can include, but is not limited to intense, high sunlight, high altitudes, radioactive underground/enclosed mining sites, and extraterrestrial environments. It is suggested that melanin containing gels and dried gels can be used for radiation protection including, but not limited to, aeronautical suits, vehicles, space craft and gear/equipment; patient, clinician, and/or technician, miners, for uses in radioprotective gear; drapes, blanket, patient wrap blanket, table drape scatter shield, garments, including, but not limited to a suit, robe, lab coat, apron, thyroid collar, shirt, blouse, t-shirt, top, trousers, shorts, breast guard, abdominal guard, pregnancy lap guard, brachytherapy guard, gonad shield, head cap, head hat, arm guards, leg guards, shin guards, headband, gloves, and the like, as well as incorporation into image intensifier drape, as known to the skilled artisan.

In another embodiment, 4.0 grams of loose melanin powder, at a uniform depth of 1 cm, were placed in 3 cm diameter test Petri dishes. This corresponded to a concentration of 565 mg/cm² of melanin. Control Petri dishes remained empty, lacking melanin powder. Both the test and control Petri dishes were subjected to Cobalt 60 gamma radiation of 1.33 MeV and 1.17 MeV, to result in 200 Gy exposure. Based on the equation:

I=Iₒe^{-μx},

Where: I=the intensity of photons transmitted across some distance x

I₀=the initial intensity of photons

e=Natural log

μ=the linear attenuation coefficient

x=distance traveled

it was estimated that the melanin powder had an attenuation coefficient of 0.1, blocking 10% of the Cobalt 60 gamma radiation at a thickness of 1 cm.

Based on this information, the radiation blocking properties of melanin as a powder at various concentrations can be estimated. Powdered melanin at a thickness of 23 cm can block 99.999% of the radiation at this energy level, while a thickness of about 2.3 cm has just 50% of the effectiveness of a thickness of 6.8 cm lead for 99.999% attenuation. Alternatively, just enough melanin paint to block 50% of the radiation may be used to replace much of lead needed for complete blockage, leading to a lighter weight material that, at a combination of just 2.3 cm melanin paint+1.1 cm thickness lead will block 99.999% of Cobalt 60 gamma radiation. The results are presented Table 2.

TABLE 2

| | | Expected | |
|---|---|---|---|
| | Required | cm | Required |
| | thickness of | thickness of | thickness |
| % | melanin | melanin | of lead in |
| Attenuation | powder in cm | paint | cm |
| 10 | 1.05 | | |
| 25 | 2.88 | 1 | |
| 50 | 6.93 | 2.3 | 1.1 |
| 75 | 13.86 | 4.7 | |
| 99.999 | 70 | 23 | 6.8 |

### Example 7: Layering Gel Sheets with Melanin and Adding Melanin Powder Between Gels Sheets

The melanin gel can be prepared as described in Example 6, dried to resemble a plastic and placed into a 9 cm Petri dish. Additional dried melanin layers can be added by either sprinkling and/or spraying a melanin powder between the layers. An adhesive, mordant, binder, as known to the skilled artisan can be incorporated into the powdered melanin to adhere the powdered melanin to each layer. Employing multilayers of the melanin gel in the plastic film with or without additional melanin powder interleaved between each plastic layer can be expected to reach a combined level of between 60 to 80 mg/cm² melanin which could be capable of completely blocking X-ray radiation (60/3 mg/cm²=20 layers, 20×4% X-rays blocked/layer=80% X-ray radiation blocked while approximately 25 layers×4% X-rays blocked/layer=100% X-rays blocked). The dried gel, having 1 to more than 10 melanin gel layers at a thickness of about 0.1 to about 4 cm would have a final thickness of about 5 cm but at less than half the weight of a lead shielding providing comparable shield.

### Example 8: Novel Process for Nanomelanin Production

Nanoparticles of melanin are known to be more effective in blocking ionizing radiation and can be easily generated in large quantities using methods as disclosed herein by Applicants from aqueous solutions of melanin as the melanin raw material.

To produce aqueous solutions of melanin the fungus was grown in a liquid culture medium similar to that mentioned in Indian Patent No. 285818 (Dated: Dec. 4, 2019, incorporated by reference). It comprised Glucose: about 1.0%; Peptone: about 1.0%; Yeast Extract: about 0.1%; Copper Sulfate: about 0.001%; Iron sulfate: about 0.01%; Magnesium sulfate: about 0.02%; Dipotassium hydrogen phosphate: about 0.05%; Tyrosine: about 1.0%. The cultures were grown on a shaker at 200 rpm at approximately 28° C.

After growth for about five days, the melanin was recovered by spray-drying the black culture filtrate, instead of precipitating with acid or alum. The yield of melanin was found to exceed 7 gm/L and up to a total of at least 8 gm/L. This melanin had the added advantage of being readily soluble in water. While the earlier process recovered the dissolved melanin from the culture filtrate by precipitating with acid and then redissolving in NaOH of pH 12.0, the spray-dried process produced a melanin that readily dissolved in water without any further treatment. The solubility of the melanin was also very high. Thus, up to 4 gm of melanin per 100 ml was dissolved in pH 7.0 water. Melanin solubility increased with pH and a total of 7 gm of melanin dissolved in water adjusted to about pH 8.0 and about pH 9.0 as illustrated in the Solubility plot in FIG. 7.

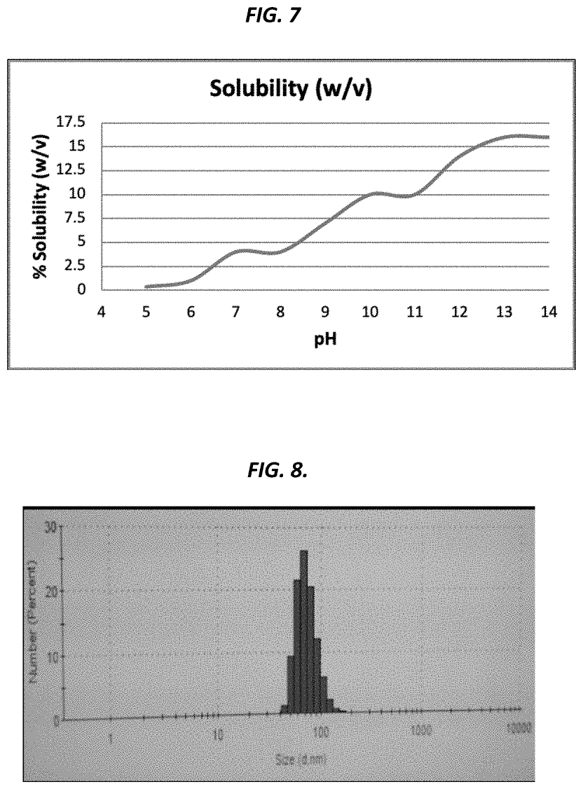

FIG. 8 illustrates the size distribution of nanomelanin obtained by DLS analysis which ranged predominantly from about 80 nm to about 100 nanometres. Therefore, Applicants' method not only yielded a large quantity of melanin it also produced a melanin that was highly soluble. Additionally, the method required fewer processing steps and fewer raw materials to complete the process establishing the method as an economical and efficient way to produce melanin and at an industrial scale.

### Example 9: Preparation of Aqueous Dispersion of Melanin Nanoparticles

An aqueous dispersion of melanin nanoparticles was prepared using two different surfactants videlicet Cetyl trimethyl ammonium bromide (CTAB) and Alcohol ethoxylate.

In one embodiment, 10 mg melanin powder was dissolved in 10 ml distilled water at about pH 7.0. A solution of 0.1 N NaOH was added to the solution to increase the pH of the resulting solution up to about 11.0. CTAB was added to the solution, for a final concentration of CTAB of about 1.5 mM-to-2.0 mM. This was followed by adding 0.1 N HCl dropwise while sonicating/stirring (at 750 rpm) the solution on an ice bath until pH drops to about pH 7.0. The resulting solution was then centrifuged at 9500 rpm for 15 min. The supernatant was decanted, and the precipitate was redispersed in distilled water at about pH 7.0 after washing 3 times with distilled water. It was observed that only a small amount of melanin was settled after centrifugation. The precipitate was redispersed in water. Both the redispersed precipitate and the supernatant were analyzed for size of the nanoparticles using the DLS analyzer (Zetasizer Nano S, Melvern Instruments Limited, Worcestershire, UK). Most of the nanoparticles are present in the supernatant.

In another embodiment, 1.0 gm melanin powder was dissolved in 100 ml distilled water at about pH 7.0. About five to about ten drops of alcohol ethoxylate surfactant were added to the solution while stirring continuously. Since the pH of the solution was neutral, HCl was not added to the solution. The solution was then centrifuged at 9500 rpm for 15 min. The supernatant was decanted, and the precipitate was redispersed in distilled water at about pH 7.0 after washing 3 times with distilled water. Both the redispersed precipitate and the supernatant were analyzed for size of the nanoparticles using the DLS analyzer. Most of the nanoparticles are present in the supernatant.

### Example 10: Organosol Preparation

There is a need in the art for a melanin that is easily miscible with organic solvents, e.g., oil-based spray paints for use in preventing ionizing radiation penetration. Thus, Applicants have found an alternative to water soluble melanin. Applicants have developed a form of Nanomelanin that was ‘synthesized’ by generation through an organosol preparation and was found to be easily dispersed in organic solvents including, but not limited to, Toluene, 2-Propanol, Tetrahydrofuran, Chloroform, Ethyl acetate and Dimethyl sulfoxide. Stearyl amine was used as a phase transfer molecule. 50 ml 0.5% aqueous melanin solution was added to 100 ml 10 mM stearyl amine in chloroform. 0.1 N HCl was added dropwise to the solution while stirring at 750 rpm. The final pH of the mixture was in the range of about pH 2.0 to about pH 3.0. The resulting solution was stirred at 750 rpm while maintaining the temperature at 37° C. for 4 hours and then transferred to a separating funnel. After complete phase separation, the lower, organic phase was collected, and the melanin containing upper, aqueous phase was transferred to a flask. This procedure was repeated three times to ensure complete phase transfer of melanin. The aqueous phase was either spray-dried, drum dried or freeze-dried to obtain the nanoparticles The final particle size of the organic-dispersible melanin nanoparticles was in the range of about 10 nm to about 15 nm. Furthermore, the dispersibility of the melanin nanoparticles was tested in various solvents such as Toluene, 2-Propanol, Tetrahydrofuran, Chloroform, Ethyl Acetate and Dimethyl Sulfoxide. The melanin nanoparticles were dispersible in each of these solvents (data not shown).

Applicants have developed a melanin that is soluble in water and a melanin that is dispersible in organic solvents. Either form of melanin can be spray-dried or freeze-dried and stored as a powder. The melanin powder can be resolubilized or redispersed in each powder's respective solvent. As illustrated and described supra, the examples demonstrate that melanin nanoparticles for use in radiation shielding can be easily generated directly from an aqueous melanin solution.

### Example 11: Melanin Slurries as Applied Coatings

Melanin in the form of a slurry can be used to block ionizing radiation in a coating applied to metals, instruments, walls, containers and the like. Such a slurry will be mostly in the form of at least 90 to 99% melanin powder, while a water or cream vehicle would comprise about 10% to about 1% of the total weight of the slurry. The slurry, when packed into a column of 1 cm height, will block at least 20% of X-rays at 80 keV and at least 10% of gamma radiation generated from a Cobalt 60 gamma radiation source (Gamma Cell C-20), and potentially more than about 20% of X-rays and more than about 10% of gamma radiation. This source of radiation simultaneously gives off two gamma rays—1.33 MeV and 1.17 MeV of per each atom decay. Thus, if 20% or more of lead or other metal is replaced by a melanin slurry as disclosed herein, the overall weight of the radiation barrier is comparably reduced.

### Example 12: Nanomelanin Coating in an Industrial Applications

A coating made up of melanin nanoparticles can be used for blocking ionizing radiation. The coating can contain about 10% to about 99% by weight of nanoparticles of melanin, with a binder or a paint at the concentration of about 90% to about 1%. The thickness of the coating will vary with the amount of shielding required. The thickness could range from about 1 mm to about 2 cm. Such a shielding can block at least 70% of X-ray at 80 keV and at least 50% of gamma rays from a Cobalt 60 gamma radiation source. Possible Industrial setting include, but are not limited to commercial isotope manufacturing, deep earth uranium and plutonium mining, aeronautical and space travel, medical diagnostics.

Ideally, a coating of melanin nanoparticles in a textile, garment, apparel, protective personal gear should be able to block ionizing radiation with a thickness not exceeding 1.0 cm. These protective textiles, wearables and gear should effectively protect against lower energy radiation, such as X-rays of about 40 keV to about 200 keV. The thickness could be several centimetres in the case of a metal surface, container, spacecraft, medical devices etc. that will be exposed to high levels of gamma radiation energy in the range of about several hundreds of keV as well as about 1 MeV to about 2 MeV.

A coating of lead plus melanin solution/melanin powder/melanin nanoparticles in various ranges will bring down the total weight of the protective coating when compared to use of lead alone.

The thickness of a coating comprising nanoparticles will be much less than that comprising melanin solution and melanin powder, being only about a few millimetres to about a centimeter thick for uses that will require blocking X-rays and low energy gamma rays.

While the principles of this invention have been described in connection with specific embodiments, it should be understood clearly that these descriptions are made only by way of example and are not intended to limit the scope of the invention. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

1. A radioprotective composition comprising:
a) At least 0.1 to at least 8.0% (wt./vol.) melanin(s); and
b) 0.1 to 97% solvent(s);
d)
wherein at least one melanin is an aqueous soluble melanin, and wherein incorporation of said composition into a material, clothing, barrier or coating renders the material, clothing, barrier or coating radioprotective.

2. The radioprotective composition of claim 1, wherein the aqueous soluble melanin is derived from a fungal, bacterial, plant, cellular and/or a synthetic melanin and combinations thereof.

3. The radioprotective composition of claim 1, wherein the aqueous soluble melanin is at least one of a eumelanin, pheomelanin, allomelanin and DHN-melanin and combinations thereof.

4. The radioprotective composition of claim 1, wherein the solvent(s) is at least one of water, acetone, dimethylformamide (DMF), acetonitrile (MeCN), dimethyl sulfoxide (DMSO), nitromethane, propylene carbonate, formic acid, n-Propanol, ethanol, methanol, nitrocellulose lacquer, and combinations thereof.

5. The radioprotective composition of claim 1, further comprising 0.1 to 30% binder selected from is-one or more of an alkyd resin, an acrylic resin, a vinyl-acrylic resin, a vinyl acetate/ethylene (VAE) resin, a polyurethane, polyester, a melamine resin, epoxy, silanes, siloxane or oil and combinations thereof.

6. The radioprotective composition of claim 1, further comprising 0.1 to 50% additive; wherein the additive affects at least one of the radioprotective composition's pigment stability, antifreeze properties, foaming and combinations thereof.

7. The radioprotective composition of claim 2, wherein the aqueous soluble melanin is derived from a fungus, wherein the fungus is one or more of Gliocephalotrichum simplex, Gliocephalotrichum sp. MTCC 5489, Cladosporium sp., Alternaria sp., Curvularia sp., Chaetomium sp., Aspergillus sp., and combinations thereof.

8. (canceled)

9. The radioprotective composition of claim 1, wherein the composition is incorporated into a coating selected from a paint, dye, gel, spray, paste, foam, liquid, cream, lotion, sizing agent, water-proofing agent, and combinations thereof.

10. The radioprotective composition of claim 9, wherein the coating attaches to a material selected from a metal, a naturally sourced textile, a synthetic textile, fruit, vegetable, edible produce.

11.-18. (canceled)

19. The radioprotective composition of claim 1, wherein the radioprotective coating comprises a paint, dye, adhesive, gel, spray, paste, foam, liquid, cream, lotion, sizing agent, water-proofing agent and combinations thereof.

20. The radioprotective composition of claim 10, wherein the radioprotective coating comprises coating the material with one or more layers of the coating.

21. The radioprotective composition of claim 20, wherein a plurality of layers of the radioprotective coating comprises about 0.2-about 20% (wt./vol.) melanin.

22. The radioprotective composition of claim 20, wherein the coating contains about 1 to about 364 mg/cm² of melanin.

23.-26. (canceled)

27. The radioprotective composition of claim 21, wherein the radioprotective coating adheres to the material by air drying, mordant, binder, heating, polymerization, covalent bonding.

28. The radioprotective composition of claim 27, wherein the coating is attachable to the material selected from a metal, a naturally sourced textile, a synthetic textile, latex, rubber, fruit, vegetable, edible produce.

29.-33. (canceled)

34. The radioprotective composition of claim 20, wherein the coating decreases one or more of photo-bleaching, color fading, degradation, UV exposure of the material.

35. (canceled)

36. An improved process for producing aqueous soluble melanin of claim 1 comprising:
a) Inoculating a fungal culture into a liquid culture medium;
b) Incubating the fungal culture medium at about 28° C., with shaking at 200 rpm for about 3-6 days; and
c) Collecting a black culture filtrate from the liquid culture medium:
wherein the melanin yield was about 7 gm/L to about 8 gm/L, wherein the improved process produced large quantities of aqueous, highly soluble melanin, and wherein the aqueous soluble melanin can be used directly for generation of melanin nanoparticles.

37.-38. (canceled)

39. The process of claim 36, wherein the fungus secretes the aqueous soluble melanin into the culture medium, wherein the melanin is in a dissolved state.

40.-42. (canceled)

43. The process of claim 39, wherein the aqueous soluble melanin secreted into the culture medium is used directly for generation of organic-dispersible melanin nanoparticles.

44.-46. (canceled)

47. The process of claim 43, wherein the organic-dispersible melanin nanoparticles are about 20 nm to about 200 nm.

48.-50. (canceled)
